(19) United States
(12) Patent Application Publication
(10) Pub. No.: US 2022/0361851 A1
(43) Pub. Date: Nov. 17, 2022

(54) DEVICE AND CONTROL PROGRAM

(71) Applicant: GE Precision Healthcare LLC, Wauwatosa, WI (US)

(72) Inventor: Naohisa Kamiyama, Kunitachi (JP)

(21) Appl. No.: 17/744,309

(22) Filed: May 13, 2022

(30) Foreign Application Priority Data
May 13, 2021 (JP) 2021-081360

(51) Int. Cl. A61B 8/08; A61B 8/14; A61B 8/00

(52) U.S. Cl. CPC A61B 8/5269; A61B 8/14; A61B 8/565; A61B 8/54

(57) ABSTRACT

A device and a control program of a device. According to an embodiment, the device includes a display and a processor. The processor is configured to display first and second B-mode images created based on an echo signal of an ultrasonic pulse acquired from a subject on the display. The first and second B-mode images displayed on the display are moving images, and the first and second B-mode images of each frame forming the moving image are created based on the same echo signal. The second B-mode image is displayed smaller than the first B-mode image on the display.

### CROSS-REFERENCE TO RELATED APPLICATIONS

This specification is based upon and claims the benefit of priority from Japanese patent application number JP 2021-081360 filed on May 13, 2021, the entire contents of which are incorporated herein by reference.

### TECHNICAL FIELD

The present invention relates to a device that displays a B-mode image and a control program thereof.

### BACKGROUND

In recent years, the number of severe fatty liver and nonalcoholic steatohepatitis (NASH) diseases has increased, and early detection of these diseases is desired. Ultrasound is a suitable modality for the diagnosis and follow-up of the aforementioned diffuse disease due to its simplicity and ability to perform frequent examinations.

A bamboo screen pattern is an acoustic shadow created in a sound ray direction of ultrasonic waves due to refraction of the ultrasonic waves. The refraction is caused by a boundary between the liver parenchyma and intravascular blood. In the case of fatty liver, fat droplets accumulate in the liver and the sound propagation speed in the liver parenchyma is reduced; therefore, this acoustic shadow becomes more intense because the sound propagation ratio to that of the blood vessels is greater. The bamboo screen pattern also arises from microvascular cross-sections, resulting in a three-dimensional, widespread, raindrop-like pattern, in other words, a stripe pattern. The bamboo screen pattern is also called a bamboo screen echo.

Ultrasound diagnostic devices are provided with various functions for improving diagnostic imaging performance. Many are intended to improve image quality and ease of inspection, and are functions that are used all the time rather than being used in specific cases. However, it has become clear that some are counterproductive in terms of visibility of the bamboo screen pattern, although they contribute to improving the visibility of structures and the like.

In order to improve the visibility of the bamboo screen pattern, it is conceivable to create a B-mode image without using the aforementioned functions, which are always used for the purpose of improving image quality and the like. However, this sacrifices the image quality required for diagnostic imaging for B-mode images, in addition to the visibility of bamboo screen patterns, such as the visibility of a structure and the like. Therefore, while improving the visibility of the bamboo screen pattern, it is necessary to ensure image quality from a perspective other than the visibility of the bamboo screen pattern, such as the visibility of a structure and the like.

### BRIEF SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

A device including a display and a processor. The processor is configured to display first and second B-mode images created based on an echo signal of an ultrasonic pulse acquired from a subject on the display. The first and second B-mode images displayed on the display are moving images, and the first and second B-mode images of each frame forming the moving image are created based on the same echo signal. The second B-mode image is displayed smaller than the first B-mode image.

A device including a display and a processor. The processor is configured to display a first B-mode image and a second B-mode image of a subject on the display. The first B-mode image is a B-mode image created based on a first echo signal acquired by transmitting a first ultrasonic pulse to the subject under a first transmission condition. The second B-mode image is a B-mode image created based on a second echo signal acquired by transmitting a second ultrasonic pulse to the subject under a second transmission condition, and the second transmission condition includes a condition in which a plurality of acoustic shadows extending in a sound ray direction in the B-mode image are emphasized as compared to the first transmission condition. The first and second B-mode images are moving images, and the first and second B-mode images of each frame forming the moving image are created based on the first and second echo signals forming temporally adjacent frames. The second B-mode image is displayed smaller than the first B-mode image.

A control program of a device including a display and a processor. The control program is configured to cause the processor to execute control, which includes displaying first and second B-mode images created based on an echo signal of an ultrasonic pulse acquired from a subject on the display. The first and second B-mode images displayed on the display are moving images, and the first and second B-mode images of each frame forming the moving image are created based on the same echo signal. The second B-mode image is displayed smaller than the first B-mode image.

### BRIEF DESCRIPTION OF DRAWINGS

FIG. 1 is a block diagram illustrating an example of an ultrasound diagnostic device according to an embodiment;

FIG. 2 is a diagram illustrating a display on which an image containing a first B-mode image and a second B-mode image is displayed according to an embodiment;

FIG. 3 is an example of a flowchart showing a process according to an embodiment;

FIG. 4 is a diagram for describing creation of the first and second B-mode images according to an embodiment;

FIG. 5 is a diagram for describing an example of a moving image according to an embodiment;

FIG. 6 is a diagram for describing creation of first and second B-mode images according to an embodiment;

FIG. 7 is a diagram for describing an example of a moving image according to an embodiment;

FIG. 8 is a diagram for describing an example of a moving image according to an embodiment;

FIG. 9 is a block diagram illustrating an example of a system according to an embodiment; and

FIG. 10 is an example of a flowchart showing a process according to an embodiment.

### DETAILED DESCRIPTION

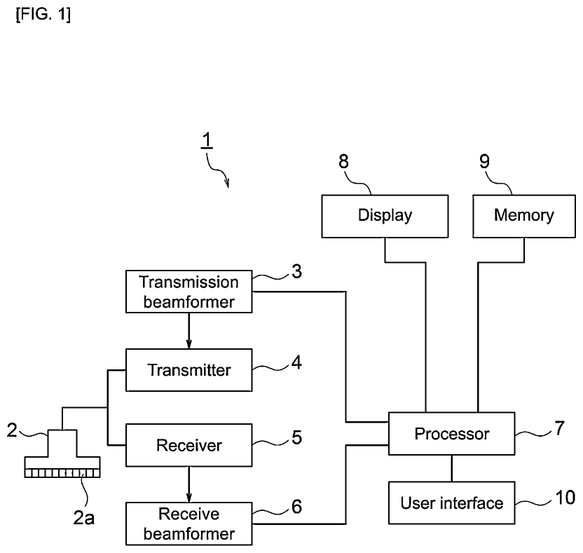

First, Embodiment 1 will be described. An ultrasound diagnostic device 1 illustrated in FIG. 1 includes an ultrasound probe 2, a transmission beamformer 3, and a transmitter 4. The ultrasound probe 2 performs ultrasound scanning on a subject, and receives an ultrasonic echo signal.

More specifically, the ultrasound probe 2 has a plurality of vibrating elements 2a that emit pulsed ultrasonic waves to the subject (not illustrated in the drawings). The plurality of vibrating elements 2a are driven by the transmission beamformer 3 and transmitter 4 to emit pulsed ultrasonic waves. The vibrating element 2a is a piezoelectric element.

The ultrasound diagnostic device 1 further includes a receiver 5 and a receive beamformer 6. The pulsed ultrasonic waves emitted from the vibrating elements 2a is reflected within the subject to generate echoes that return to the vibrating element 2a. The echo is converted into an electrical signal by the vibrating element 2a to become an echo signal, which is input to the receiver 5. The echo signal is amplified and the like by a required gain in the receiver 5 and then input to the receive beamformer 6, and receive beamforming is performed in the receive beamformer 6. The receive beamformer 6 outputs ultrasound data after receive beamforming.

The receive beamformer 6 may be a hardware beamformer or a software beamformer. If the receive beamformer 6 is a software beamformer, the receive beamformer 6 may include one or a plurality of processors, including any one or more of a graphics processing unit (GPU), a microprocessor, a central processing unit (CPU), a digital signal processor (DSP), or other types of processors capable of performing a logical operation. A processor configuring the receive beamformer 6 may be configured by a processor different from a processor 7 to be described later or may be configured by the processor 7.

The ultrasound probe 2 may include an electrical circuit for performing all or a portion of transmission beamforming and/or receive beamforming. For example, all or a portion of the transmission beamformer 3, the transmitter 4, the receiver 5, and the receive beamformer 6 may be provided in the ultrasound probe 2.

The ultrasound diagnostic device 1 also includes a processor 7 for controlling the transmission beamformer 3, transmitter 4, receiver 5, and receive beamformer 6. Furthermore, the ultrasound diagnostic device 1 includes a display 8, a memory 9, and a user interface 10.

The processor 7 includes one or a plurality of processors. The processor 7 is in electronic communication with the ultrasound probe 2. The processor 7 can control the ultrasound probe 2 to acquire ultrasound data. The processor 7 controls which of the vibrating elements 2a is active and the shape of an ultrasonic beam transmitted from the ultrasound probe 2. The processor 7 is also in electronic communication with display 8, which allows the processor 7 to process ultrasound data into an ultrasound image for displaying on the display 8. The term “electronic communication” may be defined to include both wired and wireless communications. The processor 7 may include a central processing unit (CPU) according to one embodiment. According to another embodiment, the processor 7 may include another electronic component that may perform a processing function such as a digital signal processor, a field programmable gate array (FPGA), a graphics processing unit (GPU), another type of processor, and the like. According to another embodiment, the processor 7 may include a plurality of electronic components capable of performing a processing function. For example, the processor 7 may include two or more electronic components selected from a list of electronic components including a central processing unit, a digital signal processor, a field programmable gate array, and a graphics processing unit. The processor 7 may also include a complex demodulator (not illustrated in the drawings) that demodulates RF data. In another embodiment, demodulation may be performed early in the processing chain.

The processor 7 is configured to perform one or a plurality of processing operations in the data in accordance with the plurality of selectable ultrasound modalities. When echo signals are received, the data may be processed in real time during a scan session. For the purpose of this disclosure, the term “real time” is defined to include procedures that are performed without any deliberate delay.

The data may also be temporarily stored in a buffer (not shown) during scanning of the ultrasound waves and may be processed in live or off line operations rather than real time. In this disclosure, the term “data” may be used to refer to one or a plurality of data sets acquired using the ultrasound diagnostic device 1.

The ultrasound data can be processed by the processor 7 with another or different mode-related module (such as B-mode, color Doppler, M-mode, color M-mode, spectral Doppler, contrast mode, elastography, TVI, strain, strain rate, and the like) to create ultrasound image data. For example, one or a plurality of modules may generate an ultrasound image, such as B-mode, color Doppler, M-mode, color M-mode, spectral Doppler, contrast mode, elastography, TVI, strain, strain rate, combinations thereof, and the like.

An image beam and/or an image frame may be saved and timing information may be recorded indicating when the data is captured into the memory. The module may include, for example, a scan conversion module that performs a scan conversion operation to convert an image frame from a coordinate beam space to display space coordinates. A video processor module may be provided that reads the image frame from the memory while the procedure is being performed on the subject, displaying the image frame in real time. The video processor module may save the image frame in an image memory, where ultrasound images are read from image memory and displayed on the display 8.

Note that as used in the present specification, the term “image” broadly refers to both visible images and data representing visible images. Furthermore, the term “data” can include raw data, which is ultrasound data before a scanning transformation operation, and image data, which is data after the scanning transformation operation.

If the processor 7 includes a plurality of processors, the plurality of processors may be responsible for the aforementioned processing tasks assigned by the processor 7. For example, the first processor may be used to demodulate and decimate RF signals, while the second processor may be used to further process the data and then display images. Furthermore, for example, if the receive beamformer 6 is a software beamformer, a processing function thereof may be performed via a single processor or via a plurality of processors.

The display 8 is an LED (Light Emitting Diode) display, LCD (Liquid Crystal Display), OLED (Electro-Luminescence) display, or the like.

The memory 9 is an arbitrary known data storage medium. In one example, the ultrasound image displaying system 1 includes a plurality of memories 9, including non-transient and transient storage media as the memory 9. A non-transient storage medium is a non-volatile storage medium, such as a hard disk (HDD), read only memory (ROM), or the like. Furthermore, the non-transient storage medium may also include a portable storage medium such as a CD (Compact Disk), DVD (Digital Versatile Disk), or the like. A program executed by the processor 7 is stored in the non-transient storage medium. A transient storage medium is a volatile storage medium, such as RAM (Random Access Memory) and the like.

The user interface 10 may accept operator input. For example, the user interface 10 accepts an instruction and information input from an operator. The user interface 10 is configured to include a keyboard, a hard key, a trackball, a rotary control, a soft key, and the like. The user interface 10 may include a touch screen that displays a soft key or the like.

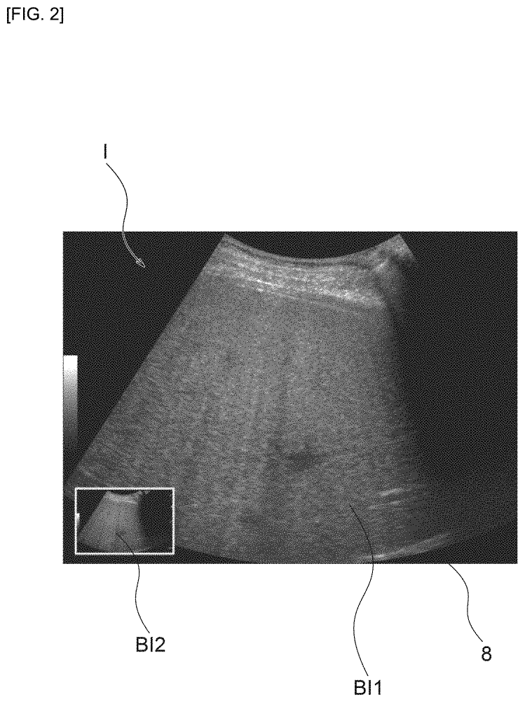

Next, a process of the present example will be described. Herein, a process of displaying an image I illustrated in FIG. 2 on the display 8 will be described. The image I includes a first B-mode image BI1 and a second B-mode image BI2. The first B-mode image BI1 and the second B-mode image BI2 are real-time moving images.

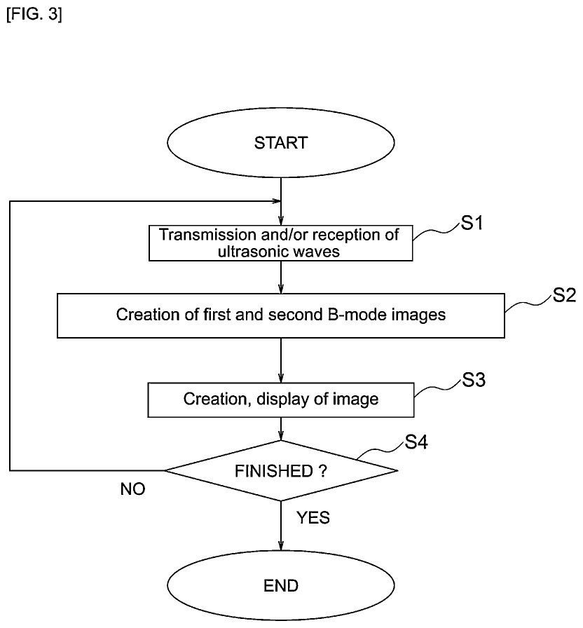

FIG. 3 shows a flowchart of the process of the present example. For example, when the user interface accepts input from an operator to start the process, the process from step S1 onward is initiated. First, in step S1, the processor 7 controls the ultrasound probe 2 to transmit an ultrasonic pulse. The ultrasound probe 2 transmits the ultrasonic pulse to the subject and receives an echo signal. Herein, the ultrasonic pulses are transmitted and received for one frame.

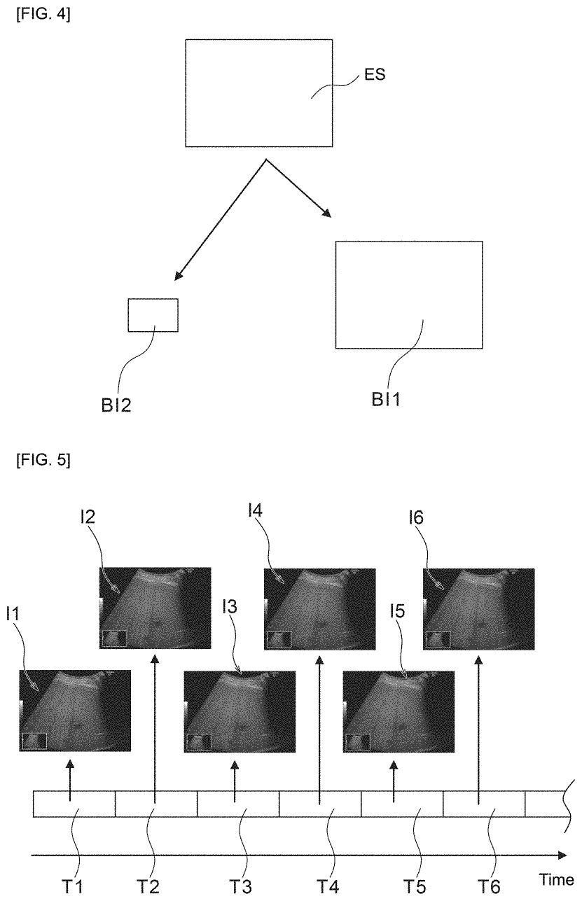

Next, in step S2, the processor 7 creates the first B-mode image BI1 and the second B-mode image BI2 for one frame based on an echo signal ES for one frame, as illustrated in FIG. 4. In FIG. 4, the echo signal ES for one frame and the first and second B-mode images BI1, BI2 are represented by rectangles for convenience. The echo signal ES was acquired in step S1. The first and second B-mode images BI1, BI2 are created based on the same echo signal ES. The echo signal ES may be a concept that includes raw data created from an echo signal received by the ultrasound probe 2.

The processor 7 creates the first and second B-mode images BI1, BI2 such that the second B-mode image BI2 appears smaller on the display 8 than the first B-mode image BI1. The sizes of the first and second B-mode images BI1, BI2 will be described in more detail. First, the first B-mode image BI1 is of normal size, and is large enough to ensure diagnostic imaging performance from a perspective other than the visibility of a plurality of acoustic shadows extending in a sound ray direction, in other words, a bamboo screen pattern. “Perspective other than the visibility of the bamboo screen pattern” includes, for example, the visibility of structures for diagnostic imaging and the like.

Next, the size of the second B-mode image BI2 will be described. In general, B-mode images are more known for their relatively low spatial frequency components when displayed small. The bamboo screen pattern is a relatively low spatial frequency component. The second B-mode image BI2 is smaller than the first B-mode image BI1 and is a size where the bamboo screen pattern is emphasized. However, the second B-mode image BI2 is large enough to recognize the bamboo screen pattern. Note that although the displayed sizes are different, the first B-mode image BI1 and the second B-mode image BI2 are images of the same portion of the subject.

The first and second B-mode images BI1, BI2 are created under the same condition. The condition may ensure image quality from a perspective other than visibility of the bamboo screen pattern, for example, visibility of a structure, while not leading to emphasis of the bamboo screen pattern. However, the condition herein does not include the size of the image.

Next, in step S3, the processor 7 creates an image I including the first B-mode image BI1 and the second B-mode image BI2, then displays the image on the display 8 as illustrated in FIG. 2. The processor 7 combines the first B-mode image BI1 and the second B-mode image to create the image I. In FIG. 2, in the image I, the second B-mode image BI2 overlaps a portion of the first B-mode image BI1. However, the positional relationship of the first and second B-mode images BI1, BI2 illustrated in FIG. 2 is an example and is not limited thereto.

Next, in step S4, the processor 7 determines whether or not to terminate the process. In one example, when a signal indicating an input to terminate the process accepted by the user interface 10 is input to the processor 7, the processor 7 determines that the process is terminated (“YES” in step S4). On the other hand, if it is determined that the process is not terminated (“NO” in step S4), the process returns to step S1 and processing is performed up to step S3, and a subsequent frame image I is displayed. In the present example, the processes of steps S1 to S4 are repeated to display a plurality of frames of the image I. The image I, in other words, the first and second B-mode images BI1, BI2 are real-time moving images. The image I may be stored in the memory 9.

To give an example, in FIG. 5, each of periods T1 to T6 represents a length of time during which ultrasonic pulses are transmitted and echo signals thereof are received for one frame. For example, one frame of the image I1 is created and displayed on the display 8 based on the echo signal obtained by transmission and reception for one frame in period T1. For the other periods T2 to T6, the images I2 to I6 are created in the same manner and displayed on the display 8. Thereby, a six-frame moving image containing of images I1 through I6 is displayed. The first and second B-mode images BI1, BI2 in each of the images I1 to I6 forming the moving image are based on the same echo signal.

However, the image I displayed on the display 8 may be an image on which a frame averaging process is performed, which is a weighted average of a plurality of frames in a time direction. The frame averaging process is performed for each of the first and second B-mode images BI1, BI2.

A reason the image I is displayed as a moving image will be described. Viewing a still image can cause visual adaptation, resulting in a decrease in the visual sensitivity of the bamboo screen pattern. Compared to a still image, it is easier to perceive the bamboo screen pattern in a moving image, where the occurrence pattern is constantly changing. Therefore, the image I including the second B-mode image BI2 is displayed as an moving image for confirming the bamboo screen pattern.

Note that in the example above, after creation and display of the first and second B-mode images BI1, BI2 of one frame are completed, the subsequent frame of ultrasonic pulses are transmitted and received, but it is not limited to this example. For example, during the process of creating and displaying the first and second B-mode images BI1, BI2, the transmission of ultrasonic pulses of the subsequent frame may be started and the reception of echo signals may be started.

According to the present example, the visibility of the bamboo screen pattern in the second B-mode image BI2 is improved based on the second B-mode image BI2 being displayed in a smaller size and being a moving image. On the other hand, in order to ensure the visibility of the bamboo screen pattern, it is not necessary to create the first and second B-mode images BI1, BI2 under a condition that sacrifices image quality from a perspective other than the visibility of the bamboo screen pattern, such as the visibility of a structure and the like. This ensures diagnostic imaging performance for the first B-mode image BI1. Therefore, the first and second B-mode images BI1, BI2 can be displayed to ensure diagnostic imaging performance while improving the visibility of the bamboo screen pattern.

Next, a modified example of Embodiment 1 will be described. First, Modified Example 1 will be described. In Modified Example 1, in step S2, the processor 7 creates the first B-mode image BI1 using the first condition. Furthermore, the processor 7 also creates the second B-mode image BI2 using the second condition different from the first condition. The second condition includes a condition that emphasizes the bamboo screen pattern in the B-mode image compared to the first condition.

For example, the first and second conditions include at least one of gain, dynamic range, and the number of frames in a frame averaging process. The gain in the second condition is lower than the gain in the first condition. The dynamic range in the second condition is lower than the dynamic range in the first condition. The number of frames in the frame averaging process in the second condition is greater than the number of frames in the first condition.

A lower gain and dynamic range emphasizes a low echo portion in a B-mode image and suppresses high echo portions, resulting in a greater emphasis on the bamboo screen pattern. Furthermore, a larger number of frames in the frame averaging process reduces the flicker noise unrelated to the bamboo screen pattern and further emphasizes the bamboo screen pattern.

Note that the first condition, as described above, may ensure image quality from a perspective other than visibility of the bamboo screen pattern, for example, visibility of a structure, while not leading to emphasis of the bamboo screen pattern.

According to the Modified Example 1, the second B-mode image created using the second condition further improves the visibility of the bamboo screen pattern.

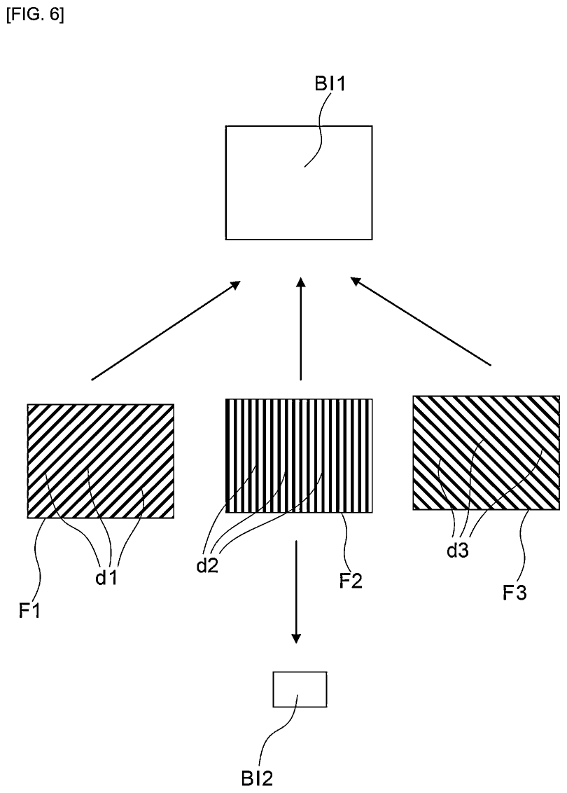

Next, Modified Example 2 will be described. In the Modified Example 2, the first B-mode image BI1 is created using a so-called transmission compounding technique. Specifically, in step S1, ultrasonic pulses are transmitted and received for a plurality of frames. A sound ray direction of the ultrasonic pulses in each of the plurality of frames are different from each other. For example, as illustrated in FIG. 6, ultrasonic pulses are transmitted and received along a first sound ray direction d1, a second direction d2, and a third direction d3 in a first frame F1, a second frame F2, and a third frame F3. The first to third directions d1 to d3 are different directions from each other.

In step S2, the processor 7 creates the first B-mode image BI1 based on echo signals configuring the plurality of frames. In the example illustrated in FIG. 6, the first B-mode image BI1 is created based on echo signals configuring the first to third frames F1 to F3. On the other hand, the processor 7 creates the second B-mode image BI2 based on the echo signals configuring one of the plurality of frames. In the example illustrated in FIG. 6, the second B-mode image BI2 is created based on echo signals configuring the second frame F2 of the first to third frames F1 to F3. Therefore, the echo signal used to create the first B-mode image BI1 and the echo signal used to create the second B-mode image BI1 contain the same echo signal.

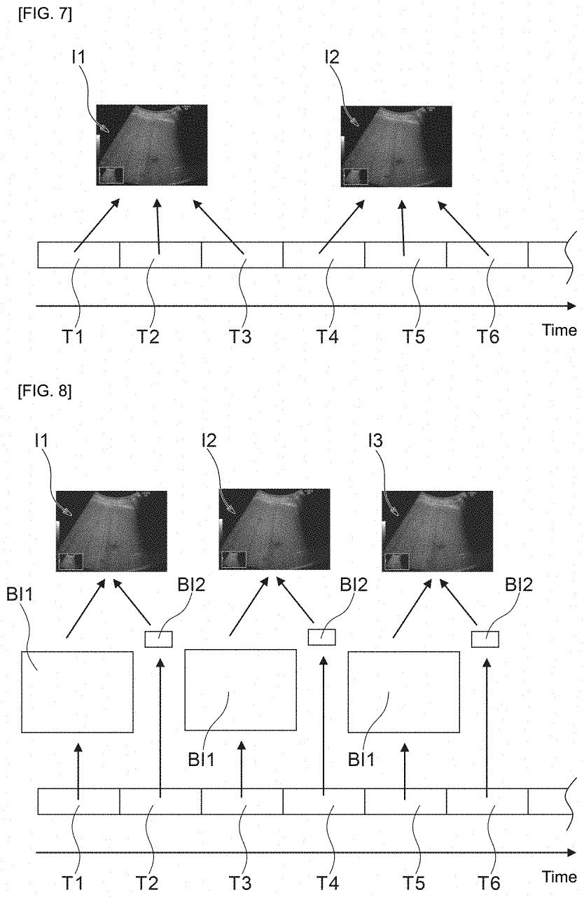

The first B-mode image BI1 and the second B-mode image BI2 are combined in step S3 to create the image I. In the Modified Example 2, as illustrated in FIG. 7, the image I1 of one frame is created based on echo signals obtained by the transmission and reception of ultrasonic pulses for three frames in the periods T1 to T3, for example. Similarly, the image I2 of a subsequent frame after the image I1 is created based on echo signals obtained by the transmission and reception of ultrasonic pulses for three frames in the periods T4 to T6. Hereinafter, the image I is created in the same manner and a moving image is displayed.

According to the Modified Example 2, the first B-mode image BI1 is an image created by a transmission compounding technique, and is therefore a more uniform image in which acoustic shadows and the like are reduced by smoothing. Thereby, visibility of a structure and the like can be more favorable and diagnostic imaging performance can be improved.

Herein, in the B-mode image, a generation direction of the bamboo screen pattern varies with a transmission direction of an ultrasonic pulse. Therefore, by creating the B-mode image using the transmission compounding technique, the bamboo screen pattern that occurs along different angles are averaged out, and the visibility of the bamboo screen pattern is reduced. However, the second B-mode image BI2 is an image created without using the transmission compounding technique and is a smaller moving image than the first B-mode image BI1, which can improve the visibility of the bamboo screen pattern.

Next, Modified Example 3 will be described. In step S1, the processor 7 controls the ultrasound probe 2 to transmit a first ultrasonic pulse under a first transmission condition to the subject, and the ultrasound probe 2 receives a first echo signal. Furthermore, in step S1, the processor 7 controls the ultrasound probe 2 to transmit a second ultrasonic pulse under a second transmission condition to the subject, and the ultrasound probe 2 receives a second echo signal. Herein, after the first ultrasonic pulse for one frame is transmitted and received, a second ultrasonic pulse for one frame is transmitted and received.

The second transmission condition includes a condition where the bamboo screen pattern is emphasized in the B-mode image as compared to the first transmission condition. In one example, a focal point in the second transmission condition is at a position closer to the ultrasound probe 2 than a focal point in the first transmission condition. In another example, the position of the focal point in the second transmission condition is closer to the ultrasound probe 2 than the position of the focal point in the first transmission condition, and yet the degree of convergence to the focal point in the second transmission condition is stronger than the degree of convergence of the focal point in the first transmission condition.

In step S2, the processor 7 creates the first B-mode image BI1 based on a first echo signal. Furthermore, the processor 7 creates the second B-mode image BI2 based on a second echo signal. For example, as illustrated in FIG. 8, the first B-mode image BI1 is created based on the first echo signal obtained by transmission and reception of the first ultrasonic pulse for one frame in the period T1. Furthermore, the second B-mode image BI1 is created based on the second echo signal obtained by transmission and reception of the second ultrasonic pulse for one frame in the period T2. Thus, the first and second B-mode images BI1, BI2 are created based on the first and second echo signals configuring temporally adjacent frames.

The first B-mode image BI1 and the second B-mode image BI2 are combined in step S3 to create the image I, which is displayed on the display 8. As illustrated in FIG. 8, the first B-mode image BI1 based on the first echo signal obtained by transmission and reception of the first ultrasonic pulse in the period T1 and the second B-mode image BI2 based on the second echo signal obtained by transmission and reception of the second ultrasonic pulse in the period T2 are combined, and an image I1 of one frame is created.

Returning to step S1, when the process is performed again up to step S3, an image I2 of a subsequent frame is created and displayed on the display 8 in the same manner. Specifically, the first B-mode image BI1 is created based on the first echo signal obtained by transmission and reception of the first ultrasonic pulse in the period T3. Furthermore, the second B-mode image BI2 is created based on the second echo signal obtained by transmission and reception of the second ultrasonic pulse in the period T4. Furthermore, the image I2 is created and displayed based on the first and second B-mode images BI1, BI2. Similarly, the first ultrasonic pulse is transmitted and received in the period T5, the second ultrasonic pulse is transmitted and received in the period T6, and an image I3 of a subsequent frame is created and displayed on the display 8. Hereinafter, the image I is created in the same manner and a moving image is displayed on the display 8.

According to Modified Example 3, the second B-mode image BI2 is created based on the second echo signal of the second ultrasonic pulse transmitted under the second transmission condition in which the bamboo screen pattern is emphasized, such that the visibility of the bamboo screen pattern can be further improved.

In Modified Example 3, similar to Modified Example 2, the first B-mode image BI1 may be created using the so-called transmission compounding technique. In this case, the first ultrasonic pulses for a plurality of frames are transmitted and received in different sound ray directions. In other words, the first transmission condition includes a condition where the first ultrasonic pulse is transmitted for a plurality of frames and the sound ray directions of the first ultrasonic pulse in each frame are different from each other. Furthermore, the first B-mode image BI1 is created based on the first echo signals configuring a plurality of frames.

Next, Modified Example 4 will be described. In Modified Example 4, the image I of a plurality of frames created in accordance with the flowchart in FIG. 3 are stored in the memory 9. Herein, the memory 9 is a non-transient storage medium, in other words, a non-volatile storage medium.

The processor 7 reads the image I from the memory 9 and then displays the image on the display 8. The image I is also a moving image and contains a first B-mode image BI1 and a second B-mode image BI2 that is smaller than the first B-mode image BI1.

In the Modified Example 4, instead of storing the image I in the memory 9, raw data obtained by transmitting and receiving ultrasonic pulses in step Si of the flowchart in FIG. 3 may be stored in the memory 9. The memory 9 stores raw data for a plurality of frames that can form a moving image. In this case, the processor 7 reads the raw data from the memory 9, creates first and second B-mode images BI1, BI2 similar to step S2, creates an image I similar to step S3, and then displays the image on the display 8.

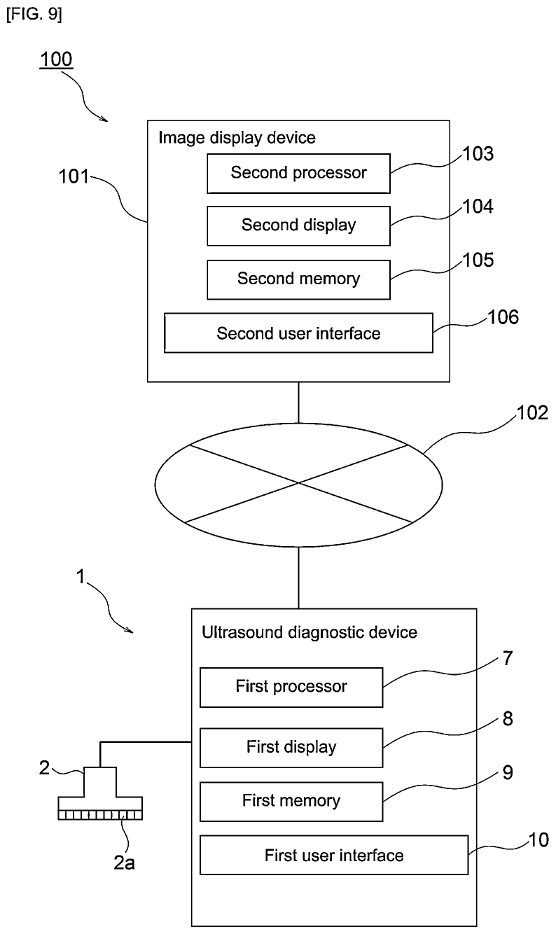

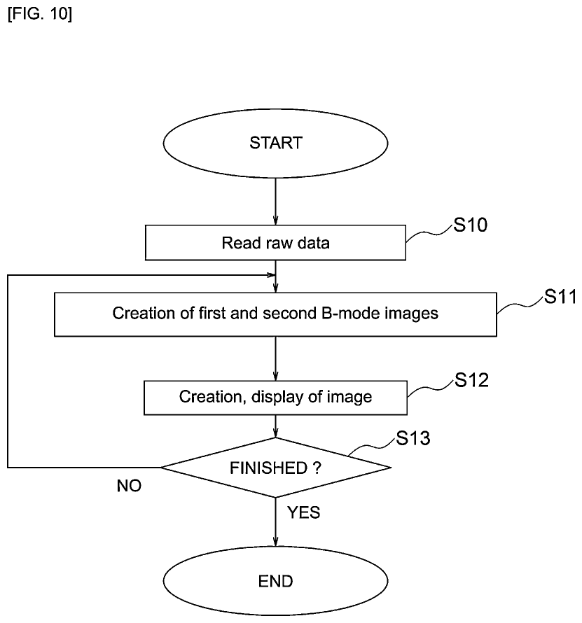

Next, Embodiment 2 will be described. A system 100 illustrated in FIG. 9 is provided with the ultrasound diagnostic device 1 and an image display device 101. The ultrasound diagnostic device 1 and the image display device 101 are connected via a network 102.

The ultrasound diagnostic device 1 has the same configuration as in FIG. 1. However, in FIG. 9, the processor 7, the display 8, the memory 9, and the user interface 10 of the ultrasound image display device 1 are described as a first processor 7, a first display 8, a first memory 9 and a first user interface 10. Although only the first processor 7, first display 8, first memory 9, and first user interface 10 are illustrated in FIG. 9 as components of the ultrasound diagnostic device 1, the ultrasound diagnostic device 1 has other components illustrated in FIG. 1. Note that each configuration is illustrated using only blocks in FIG. 9.

The image display device 101 is, for example, a workstation, portable information terminal, or the like. The image display device 101 has a second processor 103, a second display 104, a second memory 105, and a second user interface 106.

In Embodiment 2, an image I, which is a moving image, is displayed on the image display device 101. A process therefor will be described. First, when an echo signal is acquired by transmitting and receiving ultrasonic pulses in the ultrasound diagnostic device 1 similar to step S1 in FIG. 3, raw data based on the echo signal is transmitted to the image display device 101 via the network 102. Raw data is data for a plurality of frames that can form a moving image. The raw data is stored in the second memory 105. Herein, the second memory 105 is a non-transient storage medium, in other words, a non-volatile storage medium.

Next, display of the image I will be described based on the flowchart in FIG. 10. First, in step S10, the second processor 103 reads the raw data from the second memory 105. The second processor 103 reads the raw data for a plurality of frames that can form a moving image. The raw data for a plurality of frames is raw data for all frames to be displayed as a moving image.

Processes of steps S11 to S13 is the same as the processes of steps S2 to S4, except that a main body of processing is the second processor 103. If it is determined in step S13 that the process is not terminated, the process returns to step S11 and subsequent processes are performed. Thereby, the image I, including the first and second B-mode images BI1, BI2, can be displayed on the second display 104 as a moving image, which, similar to Embodiment 1, improves the visibility of the bamboo screen pattern while ensuring diagnostic imaging performance. Note that the image I may be stored in the second memory 105. Furthermore, instead of reading the raw data of all frames in step S10, one frame at a time may be read and one frame of the image I may be displayed.

Next, a modified example of Embodiment 2 will be described. In this modified example, the first processor 7 of the ultrasound diagnostic device 1 performs steps S1 to S3 in FIG. 3 to create an image I including the first and second B-mode images BI1, BI2. The image I is a moving image. Furthermore, the first processor 7 outputs the image Ito the image display device 101 via the network 102. The image I is stored in the second memory 105.

The second processor 7 reads the image I stored in the second memory 105 and displays the image on the second display 104.

In Embodiment 2, similar to Modified Example 1 of Embodiment 1, the first B-mode image BI1 may be created using the first condition and the second B-mode image BI2 may be created using the second condition. Furthermore, similar to Modified Example 2 of Embodiment 1, the first B-mode image may be created using a transmission compounding technique. Furthermore, the raw data stored in the second memory 105 may include raw data based on the first echo signal obtained by transmitting the first ultrasonic pulse and raw data based on the second echo signal obtained by transmitting the second ultrasonic pulse, similar to Modified Example 3 of Embodiment 1.

Although the present invention has been described with reference to specific embodiments, various modifications may be made and substitutions made to equivalents without departing from the scope of the present invention. In addition, many modifications can adapt specific situations or materials to the disclosure of the present invention without departing from the scope of the present invention. Therefore, the present invention is not limited to the specific embodiments that have been disclosed and is intended to include all embodiments in which the present invention falls within the scope of the appended claims.

For example, in Embodiment 2, the image I stored in the second memory 105 may be output to the ultrasound diagnostic device 1 via a network and then displayed on the first display 8.

This written description uses examples to disclose the subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A device, comprising:
a display; and
a processor, wherein the processor is configured to display first and second B-mode images created based on an echo signal of an ultrasonic pulse acquired from a subject on the display;
wherein the first and second B-mode images displayed on the display are moving images, and the first and second B-mode images of each frame forming the moving image are created based on the same echo signal; and
wherein the second B-mode image is displayed smaller than the first B-mode image.

2. A device, comprising:
a display; and
a processor, wherein the processor is configured to display a first B-mode image and a second B-mode image of a subject on the display;
wherein the first B-mode image is a B-mode image created based on a first echo signal acquired by transmitting a first ultrasonic pulse to the subject under a first transmission condition;
wherein the second B-mode image is a B-mode image created based on a second echo signal acquired by transmitting a second ultrasonic pulse to the subject under a second transmission condition, and the second transmission condition includes a condition in which a plurality of acoustic shadows extending in a sound ray direction in the B-mode image are emphasized as compared to the first transmission condition,
wherein the first and second B-mode images are moving images, and the first and second B-mode images of each frame forming the moving image are created based on the first and second echo signals forming temporally adjacent frames, and
wherein the second B-mode image is displayed smaller than the first B-mode image.

3. The device according to claim 1, wherein the first and second B-mode images are created under the same condition, and the condition is a condition that does not lead to the emphasis of a plurality of acoustic shadows extending in a sound ray direction in the first B-mode image.

4. The device according to claim 1, wherein the first B-mode image is created using a first condition, and the second B-mode image is created using a second condition, and the second condition includes a condition of emphasizing a plurality of acoustic shadows extending in a sound ray direction the second B-mode image, as compared to the first condition.

5. The device according to claim 4, wherein the first and second conditions include at least one of gain, dynamic range, and the number of frames in a frame averaging process.

6. The device according to claim 1, wherein the first B-mode image is created based on echo signals forming a plurality of frames, and sound ray directions of the ultrasonic pulse in each of the plurality of frames are different from each other, and
the second B-mode image is created based on an echo signal forming one frame of the plurality of frames.

7. The device according to claim 2, wherein the first and second transmission conditions include a focal point position and a degree of convergence to a focal point.

8. The device according to claim 2, wherein the first transmission condition includes a condition in which the first ultrasonic pulse for a plurality of frames is transmitted and sound ray directions of the first ultrasonic pulse in each of the frames are different from each other; and
wherein the first B-mode image is created based on the first echo signal forming the plurality of frames.

9. The device according to claim 1, wherein the device is an ultrasound diagnostic device, and the processor creates the first and second B-mode images.

10. The device according to claim 1, wherein the device is an image display device connected to the ultrasound diagnostic device via a network.

11. The device according to claim 10, wherein the processor creates the first and second B-mode images.

12. The device according to claim 10, wherein the ultrasound diagnostic device includes a first processor that creates the first and second B-mode images and outputs the first and second B-mode images to the image display device via the network; and
wherein the processor of the image display device is a second processor that displays, on the display, the first and second B-mode images output by the first processor.

13. A control program of a device,
the device including a display and a processor:
wherein the control program is configured to cause the processor to execute control, which includes displaying first and second B-mode images created based on an echo signal of an ultrasonic pulse acquired from a subject on the display,
wherein the first and second B-mode images displayed on the display are moving images, and the first and second B-mode images of each frame forming the moving image are created based on the same echo signal, and
wherein the second B-mode image is displayed smaller than the first B-mode image.

14. The device according to claim 2, wherein the first and second B-mode images are created under the same condition, and the condition is a condition that does not lead to the emphasis of a plurality of acoustic shadows extending in a sound ray direction in the first B-mode image.

15. The device according to claim 2, wherein the first B-mode image is created using a first condition, and the second B-mode image is created using a second condition, and the second condition includes a condition of emphasizing a plurality of acoustic shadows extending in a sound ray direction of a B-mode image, as compared to the first condition.

16. The device according to claim 2, wherein the device is an ultrasound diagnostic device, and the processor creates the first and second B-mode images.

17. The device according to claim 3, wherein the device is an ultrasound diagnostic device, and the processor creates the first and second B-mode images.

18. The device according to claim 4, wherein the device is an ultrasound diagnostic device, and the processor creates the first and second B-mode images.

19. The device according to claim 2, wherein the device is an image display device connected to the ultrasound diagnostic device via a network.

20. The device according to claim 3, wherein the device is an image display device connected to the ultrasound diagnostic device via a network.
